(19) United States
(12) Patent Application Publication
(10) Pub. No.: US 2006/0058389 A1
(43) Pub. Date: Mar. 16, 2006

(54) Novel compounds that inhibit factor xa activity

(75) Inventors: Robert Eckl, Munchen (DE); Silke Schabbert, Munchen (DE); Thilo Fuchs, Munchen (DE)

Correspondence Address: SONNENSCHEIN NATH & ROSENTHAL LLP;FOR PAULA EVANS, P.O. BOX 061080, CHICAGO, IL, 60606-1080, US

(73) Assignee: Morphochem Aktiengesellschaft fur Kombinatorische Chemie, Munchen (DE)

(21) Appl. No.: 10/501,931

(22) PCT Filed: Jan. 31, 2003
(86) PCT No.: PCT/EP03/01012

§ 371 Date: Jul. 5, 2005

(30) Foreign Application Priority Data
Jan. 31, 2002 (DE) 102 04 072.9

(51) Int. Cl. A61K 31/195; C07C 235/84

(52) U.S. Cl. 514/561; 562/450

(57) ABSTRACT

The present invention relates to compounds of formula (I): 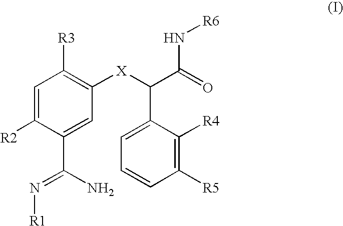 or a pharmaceutically acceptable salt, solvate, hydrate or pharmaceutically acceptable formulation thereof. Those compounds can be used in inhibiting factor Xa and in the prevention and/or treatment of thromboembolic conditions.

The present invention relates to new compounds having an inhibitory action on blood clotting (so-called anticoagulants) and to their pharmacologically acceptable salts and solvates and hydrates, to pharmaceutical compositions comprising them as active ingredient, to processes for the preparation of such compounds, salts and compositions, and to the use thereof in the prevention and/or treatment of thromboembolic conditions. Those compounds, salts and compositions are very effective factor Xa inhibitors. The present invention relates also to pro-drugs, optically active forms, racemates and diastereoisomers of those compounds and salts.

Thromboembolic conditions are caused by an increased tendency to blood clotting in people with risk factors, such as, for example relatively major operations, prolonged immobilisation, fractures of the lower extremities, obesity, blood fat metabolism disorders, infections with gram-negative organisms, cancer and older age.

Venous thromboses may lead to the development of oedema or inflammation of the tissue drained by the affected vein. Thrombosis of a deeper vein (so-called deep vein thrombosis) may lead to serious complications, such as, for example, pulmonary embolism. Arterial thrombosis may lead to ischaemic necrosis of the tissue supplied by the affected artery, such as, for example, to myocardial infarct in the case of an affected coronary artery. Other thromboembolic conditions are, for example, arterio-sclerosis, apoplexy (stroke), angina pectoris, intermittent claudication.

Under normal physiological conditions, natural blood clotting protects against major blood loss from a damaged blood vessel. During blood clotting, liquid blood is converted into a blood clot, a gelatinous mass which seals injured blood vessels by forming a plug. In that process, soluble fibrinogen present in the plasma is converted into the fibrous-gelatinous clotting substance fibrin in a multi-stage process, the so-called coagulation cascade.

A distinction is made between two different pathways of coagulation activation. The intrinsic coagulation pathway is initiated when blood comes into contact with non-physiological surfaces. The extrinsic coagulation pathway is initiated by injury to blood vessels. Both coagulation pathways join in a common pathway in which the coagulation factor X, a serine protease, is converted into its active form (factor Xa). Factor Xa, together with factor Va and Ca²⁺ in the so-called prothrombinase complex, causes prothrombin to be converted into thrombin which in turn, by cleaving peptides from fibrinogen, releases fibrin monomers, which are capable of coagulating to form fibrin fibres. Finally, factor XIII brings about cross-linking and thus stabilisation of the fibrin fibres.

Anticoagulants are used both for the prevention and for the treatment of thromboembolic conditions. As far as anticoagulants in the narrower sense are concerned, a distinction is made between heparin, which is immediately effective and which directly inhibits certain blood clotting factors, and vitamin K antagonists (for example, coumarin derivatives). The latter inhibit the production in the liver of certain clotting factors which is dependent on the presence of vitamin K, and begin to take effect only slowly. Other anticoagulant agents are the fibrinolytics, which bring about direct or indirect activation of the fibrinolytic system, and thrombocyte aggregation inhibitors, such as, for example, acetylsalicylic acid. A more seldom used method is reduction of the fibrinogen level in the blood by the enzyme ancrod. The object of using anticoagulant agents is to prevent the development of a blood clot that could close a vessel or also to dissolve it again once it has formed.

The above-mentioned anticoagulants in the narrower sense, that is to say heparin and vitamin K antagonists, have disadvantages. In the case of heparin, a distinction is made between unfractionated heparin (UFH) and low-molecular-weight heparin (LMWH). A disadvantage with UFH is the fact that it generally has to be administered intravenously, has a varying anticoagulant effect and therefore necessitates frequent monitoring of the patient and adaptation of the dosage. Although LMWH can be used subcutaneously in a constant, unmonitored dosage, its effect, compared to that of UFH, is greatly reduced because of its short chain length.

The vitamin K antagonists such as, for example, warfarin exhibit degrees of activity that differ from patient to patient, presumably owing to genetic factors. In addition to the slow onset of action mentioned above, this is associated with the disadvantage that patients have to be monitored and individual adaptation of the dosage is required.

Other known anticoagulants belong to the group of the thrombin inhibitors. Current overviews of relevant research activity in that field can be found, for example, in Jules A. Shafer, Current Opinion in Chemical Biology, 1988, 2: 458-485, Joseph P. Vacca, Current Opinion in Chemical Biology, 2000, 4: 394-400 and also in Fahad Al-Obeidi and James A. Ostrem, DDT, Vol. 3, No. 5, May 1998: 223-231.

A crucial disadvantage of thrombin inhibitors is that, in order to obtain the desired effect, it is necessary to suppress thrombin activity in vivo to such a great extent that the tendency to haemorrhage may increase, which makes dosage difficult.

In contrast, factor Xa inhibitors cause suppression of the new formation of thrombin from prothrombin, whereas they do not impair existing thrombin activity which is necessary for primary haemostasis.

The spectra of action and side-effects of some of those factor Xa inhibitors have not yet been fully investigated.

An object of the present invention was to provide new compounds having useful properties, especially an anticoagulating action.

More precisely, the object was to provide new factor Xa inhibitors having improved efficacy, reduced side-effects and/or increased selectivity. In addition, suitable pharmaceutical compositions were to be provided. Those compounds and compositions were to be administrable preferably parenterally or orally, especially orally.

A further object of the present invention was to provide a process for the preparation of those new compounds.

Those new compounds were furthermore to be suitable for use in the prevention and/or treatment of thromboembolic conditions.

The present invention describes anticoagulant compounds, their pharmacologically acceptable salts and solvates and hydrates and formulations that have a high activity and selectivity and can be administered orally. The present invention further relates to pro-drugs, optically active forms, racemates and diastereoisomers of those compounds and salts. The said compounds and salts may also themselves be pro-drugs, which are activated only by metabolisation. Pharmaceutical compositions comprising the said compounds or salts etc. as active ingredient are also described.

The present invention relates to a compound of the general formula (I): 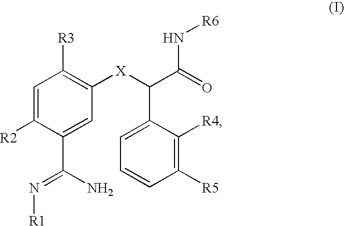
wherein
R1 is a hydrogen atom, a heteroalkyl, heteroaralkyl, heterocycloalkyl, hydroxy or alkyloxy group, R2 is a hydrogen atom or a hydroxy group, or R1 and R2 together are part of a 5- or 6-membered ring;

R3 is a hydrogen atom, a hydroxy, alkyloxy, amino, alkylamino or dialkylamino group or a halogen atom;

R4 and R5 are, each independently of the other, a hydrogen atom, a halogen atom, a hydroxy, amino, nitro or thiol group, an alkyl, heteroalkyl, aryl, heteroaryl, cycloalkyl, heterocycloalkyl, aralkyl or heteroaralkyl radical or a glycosyloxy group;

R6 is an alkyl, heteroalkyl, heteroaralkyl, heteroaryl, aralkyl, cycloalkyl, heterocycloalkyl or aryl group, R6 not being a group of formula —CHR8-CO—NR9R9′, wherein R8, R9 and R9′ are, each independently of the others, a hydrogen atom, an alkyl, heteroalkyl, heteroaralkyl, heteroaryl, aralkyl, cycloalkyl, heterocycloalkyl or aryl group, or R9 and R9′ together are part of a heterocycloalkyl or heteroaryl ring system, and

X is a group of formula NR7, O, S, SO, SO₂, SO₂NH, PO₂NH, CH₂, CHMe or CO, R7 being a hydrogen atom, an alkyl or aralkyl group;

or a pharmacologically acceptable salt, solvate, hydrate or pharmacologically acceptable formulation thereof.

There are preferably excluded compounds of formula (I) wherein R6 is an alkyl, heteroalkyl, heteroaralkyl, heteroaryl, aralkyl, cycloalkyl, heterocycloalkyl or aryl group, R6 not being a group of formula —CO—CHR8NR9R9′, wherein R8, R9 and R9′ are, each independently of the others, an alkyl, heteroalkyl, heteroaralkyl, heteroaryl, aralkyl, cycloalkyl, heterocycloalkyl or aryl group, or R9 and R9′ together are part of a heterocycloalkyl or heteroaryl ring system.

Owing to their substitution, compounds of formula (I) contain one or more centres of chirality. The present invention therefore includes both all pure enantiomers and all pure diastereoisomers and also mixtures thereof in any mixing ratio.

The expression alkyl refers to a saturated or at least partially unsaturated (for example, alkenyl, alkynyl), straight-chain or branched hydrocarbon group having 1 or 2 to 20 carbon atoms, preferably 1 or 2 to 12 carbon atoms, especially 1 or 2 to 6 carbon atoms, for example a methyl, ethyl, isopropyl, isobutyl, tert-butyl, n-hexyl, 2,2-dimethylbutyl or n-octyl group.

The expressions alkenyl and alkynyl refer to at least partially unsaturated, straight-chain or branched hydrocarbon groups having from 2 to 20 carbon atoms, preferably from 2 to 12 carbon atoms, especially from 2 to 6 carbon atoms, for example an allyl, ethynyl, propargyl, isoprenyl or hex-2-enyl group.

The expression heteroalkyl refers to an alkyl, alkenyl or alkynyl group in which one or more (preferably 1, 2 or 3) carbon atoms have been replaced by an oxygen, nitrogen, phosphorus or sulphur atom (preferably oxygen or nitrogen), for example an alkyloxy group such as, for example, methoxy or ethoxy, or a methoxymethyl, nitrile, methylcarboxyalkyl ester, carboxyalkyl ester or 2,3-dioxyethyl group. The expression heteroalkyl furthermore refers to a carboxylic acid or to a group derived from a carboxylic acid such as, for example, acyl, acyloxy, carboxyalkyl, carboxyalkyl ester, for example methylcarboxyalkyl ester, carboxyalkylamide, alkoxycarbonyl or alkoxycarbonyloxy.

The expression cycloalkyl or cyclo- refers to a saturated or partially unsaturated (for example, cycloalkenyl) group which has one or more rings forming a structure containing from 3 to 14 carbon atoms, preferably from 3 to 10 carbon atoms, for example a cyclopropyl, cyclohexyl, 1,2,3,4-tetrahydronaphthyl or cyclohex-2-enyl group.

The expression heterocycloalkyl or heterocyclo- refers to a cycloalkyl group as defined above in which one or more (preferably 1, 2 or 3) carbon atoms have been replaced by an oxygen, nitrogen, phosphorus or sulphur atom (preferably oxygen or nitrogen) and can be, for example, a piperidine, morpholine, N-methylpiperazine or N-phenylpiperazine group.

The expression aryl or Ar refers to an aromatic group which has one or more rings and is formed by a structure containing from 5 to 14 carbon atoms, preferably 5 or 6 to 10 carbon atoms, for example a phenyl, naphthyl, 2-, 3- or 4-methoxyphenyl, 2-, 3- or 4-ethoxyphenyl, 4-carboxyphenyl-alkyl or 4-hydroxyphenyl group.

The expression heteroaryl refers to an aryl group in which one or more (preferably 1, 2 or 3) carbon atoms have been replaced by an oxygen, nitrogen, phosphorus or sulphur atom (preferably oxygen or nitrogen), for example a 4-pyridyl, 2-imidazolyl, 3-pyrazolyl and isoquinolyl group.

The expressions aralkyl and heteroaralkyl refer to groups comprising, in accordance with the above definitions, both aryl or heteroaryl, respectively, and also alkyl and/or heteroalkyl and/or cycloalkyl and/or heterocycloalkyl ring systems, for example, a tetrahydroisoquinolyl, benzyl, 2- or 3-ethyl-indolyl or 4-methylpyridino group.

The expressions alkyl, heteroalkyl, cycloalkyl, hetero-cycloalkyl, aryl, heteroaryl, aralkyl and heteroaralkyl and also the expression “substituted” refer also to groups in which one or more hydrogen atoms (preferably 1, 2, 3 or 4) of such groups have been replaced by fluorine, chlorine, bromine or iodine atoms or by OH, SH, NH₂ or NO₂ groups (preferably F, Cl or OH). Those expressions refer furthermore to groups substituted by unsubstituted alkyl (preferably methyl), heteroalkyl (preferably methoxy), cycloalkyl, heterocycloalkyl, aryl, heteroaryl, aralkyl or heteroaralkyl groups.

The expressions alkylene, heteroalkylene, cycloalkylene heterocycloalkylene, arylene, heteroarylene, heteroaryl-alkylene and aralkylene refer to disubstituted alkyl, heteroalkyl, cycloalkyl, heterocycloalkyl, aryl, heteroaryl, heteroarylalkyl and aralkyl groups, that is to say to groups carrying at least two substituents other than H.

In the context of the present invention the expression “glycosyloxy group” refers to a saccharide bonded by way of an α- or β-O-glycosidic bond, especially a monosaccharide, preferably glucose or fructose.

Preference is given to compounds of the general formula (I) wherein R1 is a hydrogen atom.

Special preference is given to compounds of the general formula (I) wherein X is a group of formula NR7.

Preference is furthermore given to compounds of the general formula (I) wherein R2 is a hydrogen atom.

Preference is moreover given to compounds of the general formula (I) wherein R3 is a hydrogen atom or a hydroxy group.

Preference is furthermore given to compounds of the general formula (I) wherein R4 is a hydrogen atom, an —OH, —OCH₂COOH, —OCH₂COOCH₃, —COOH, C₁-C₄alkyloxy or glycosyloxy group or a halogen atom. Special preference is given to R4 being a β-D-glucosyloxy group.

Preference is furthermore given to compounds of the general formula (I) wherein R5 is a hydrogen atom, an —OH, —OCH₂COOH, —OCH₂COOCH₃, —COOH, C₁-C₄alkyloxy or glycosyloxy group or a halogen atom. Special preference is given to R5 being a hydrogen atom.

Preference is furthermore given to compounds of the general formula (I) wherein R6 is a group of formula -A-NR10R11, A being an alkylene, heteroalkylene, cycloalkylene, arylene, heteroarylene, heterocycloalkylene, heteroarylalkylene or aralkylene group, and R10 and R11 being, each independently of the other, a hydrogen atom, an alkyl, heteroalkyl, aryl, heteroaryl, cycloalkyl, heterocycloalkyl, aralkyl or heteroaralkyl radical or together being part of a heterocycloalkyl ring system.

Special preference is given to compounds of the general formula (I) wherein A is a para-phenylene and R10 and R11 are part of a 5- or 6-membered heterocycloalkyl ring.

Preference is furthermore given to compounds of the general formula (I) wherein R6 is a para-substituted phenyl ring.

Preference is moreover given to compounds of the general formula (I) wherein R7 is a hydrogen atom or a methyl group.

Special preference is given to compounds of the general formula (I) wherein R7 is a hydrogen atom.

Examples of pharmacologically acceptable salts of compounds of formula (I) are salts of physiologically acceptable mineral acids, such as hydrochloric acid, sulphuric acid and phosphoric acid; or salts of organic acids, such as methanesulphonic acid, p-toluenesulphonic acid, lactic acid, acetic acid, trifluoroacetic acid, citric acid, succinic acid, fumaric acid, maleic acid and salicylic acid. Compounds of formula (I) can be solvated, especially hydrated. The hydration may take place, for example, during the preparation process or as a consequence of the hygroscopic nature of the initially anhydrous compounds of formula (I).

The pharmaceutical compositions according to the present invention comprise at least one compound of formula (I) as active ingredient and optionally carrier substances and/or adjuvants.

The pro-drugs to which the present invention also relates consist of a compound of formula (I) and at least one pharmacologically acceptable protecting group that is removed under physiological conditions, for example an alkoxy, aralkyloxy, acyl or acyloxy group, such as, for example, an ethoxy, benzyloxy, acetyl or acetyloxy group.

Compounds of formula (I) according to the invention can be prepared by reaction of compounds of formulae (II), (III) and (IV) using a multi-component reaction (A. Dömling, I. Ugi, Angew. Chem. 2000, 112, 3300-3344), the radicals being defined as above. In the process, a compound of formula (II) is preferably dissolved together with a compound of formula (III) especially in a suitable solvent (preferably a mixture of acetonitrile and water) and, where appropriate, stirred (preferably for 30 minutes at room temperature). A compound of formula (IV) is then added and, where appropriate, further stirring is carried out (preferably for 15 minutes at room temperature). The optionally present solvent is then removed preferably in vacuo. The compounds prepared in the process can be purified by means of HPLC and separated into the individual stereoisomers. In the case of the compounds obtained in that manner it was found that both the compounds of formula (I) having an (R) configuration at the phenylglycine entity and also the corresponding (S)-configured compounds are very effective factor Xa inhibitors, the (S)-configured compounds having, when identically substituted, slightly better inhibitory properties. Preference is therefore given in accordance with the invention to compounds of formula (I) having an (S) configuration, whilst compounds having an (R) configuration also have very good inhibitory properties and this invention relates also thereto.

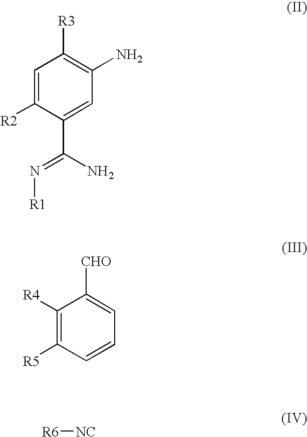

A compound or pharmaceutical composition of the present invention can be used in inhibiting factor Xa activity, in the prevention and/or treatment of thromboembolic conditions, arterial restenosis, septicaemia, cancer, acute inflammation or other conditions mediated by factor Xa activity, and especially venous thromboses, oedema or inflammation, deep vein thrombosis, pulmonary embolisms, thromboembolic complications after relatively major operations, in the case of vascular surgery, prolonged immobilisation, fractures of the lower extremities etc., arterial thromboses, especially of the coronary vessels in the event of myocardial infarct, and arteriosclerosis, stroke, angina pectoris, intermittent claudication, to mention but a few indications.

In general, as mentioned at the beginning, the active ingredients according to the invention are to have an inhibitory action towards factor Xa that is as great as. possible while having a selectivity that is as high as possible. The selectivity was assessed in the present case by comparing the inhibitory action towards factor Xa and also tryptase and thrombin (two further serine proteases).

As mentioned above, the therapeutic use of the compounds of formula (I), of their pharmacologically acceptable salts and solvates and hydrates and also formulations and pharmaceutical compositions lies within the scope of the present invention.

The present invention relates also to the use of those active ingredients in the preparation of medicaments for the prevention and/or treatment of thromboembolic conditions. In general, compounds of formula (I) are administered either individually or in combination with any other desired therapeutic agent, using the known and acceptable methods. Such therapeutically useful agents can be administered by one of the following routes: orally, for example in the form of dragees, coated tablets, pills, semi-solid substances, soft or hard capsules, solutions, emulsions or suspensions; parenterally, for example in the form of an injectable solution; rectally in the form of suppositories; by inhalation, for example in the form of a powder formulation or spray, transdermally or intranasally. For the preparation of such tablets, pills, semi-solid substances, coated tablets, dragees and hard gelatin capsules, the therapeutically usable product can be mixed with pharmacologically inert, inorganic or organic pharmaceutical carrier substances, for example with lactose, sucrose, glucose, gelatin, malt, silica gel, starch or derivatives thereof, talcum, stearic acid or salts thereof, skimmed milk powder and the like. For the preparation of soft capsules, pharmaceutical carrier substances such as, for example, vegetable oils, petroleum, animal or synthetic oils, wax, fat and polyols can be used.

For the preparation of liquid solutions and syrups, pharmaceutical carrier substances such as, for example, water, alcohols, aqueous saline solution, aqueous dextrose, polyols, glycerol, vegetable oils, petroleum and animal or synthetic oils can be used. For suppositories, pharmaceutical carrier substances such as, for example, vegetable oils, petroleum, animal or synthetic oils, wax, fat and polyols can be used. For aerosol formulations, compressed gases that are suitable for the purpose can be used, such as, for example, oxygen, nitrogen and carbon dioxide. The pharmaceutically acceptable agents may also comprise additives for preserving and stabilising, emulsifiers, sweeteners, flavourings, salts for altering the osmotic pressure, buffers, encapsulation additives and anti-oxidants.

Combinations with other therapeutic agents may comprise other active ingredients that are customarily used for the prevention and/or treatment of thromboembolic conditions, such as, for example, warfarin etc.

For the prevention and/or treatment of the conditions mentioned above, the dose of the biologically active compound according to the invention can vary within wide limits and can be adjusted to individual requirements. In general, a dose of from 0.1 μg to 10 mg/kg of body weight per day is suitable, a preferred dose being from 0.5 to 4 mg/kg per day. In suitable cases, the dose may also be below or above the stated values.

The daily dose can be administered in, for example, 1, 2, 3 or 4 individual doses. It is also possible to administer the dose for one week as a single dose.

The following Examples are intended to illustrate the invention. The stereochemistry of 3,4,5-trihydroxy-6-hydroxymethyl-tetrahydropyran-2-yloxy corresponds to that of β-D-glucose.

### EXAMPLES

General Procedure:

1 mmol of amine (II) and 1 mmol of aldehyde (III) are stirred in 20 ml of acetonitrile/water (mixing ratio of from 1:0 to 1:1) for 30 minutes at room temperature. 1 mmol of isonitrile (IV) is then added and stirring is carried out for a further 15 hours. The solvent is removed in vacuo and the residue is purified by means of HPLC.

### Example 1

### 2-(3-Carbamimidoyl-phenylamino)-N-(2-trifluoromethyl-benzyl)-2-[2-(3,4,5-trihydroxy-6-hydroxymethyl-tetrahydro-pyran-2-yloxy)-phenyl]-acetamide

C₂₉H₃₁F₃N₄O₇ (604.5882)

ESI-TOF MS: 605 [M+H]

### Example 2

### 2-(3-Carbamimidoyl-phenylamino)-N-(2,3-dihydro-benzo[1,4]dioxin-6-yl)-2-[2-(3,4,5-trihydroxy-6-hydroxymethyl-tetrahydro-pyran-2-yloxy)-phenyl]-acetamide

C₂₉H₃₂N₄O₉ (580.5998)

ESI-TOF MS: 581 [M+H]

### Example 3

### 2-(3-Carbamimidoyl-phenylamino)-N-[3-(2-oxo-pyrrolidin-1-yl)-propyl]-2-[2-(3,4,5-trihydroxy-6-hydroxymethyl-tetrahydro-pyran-2-yloxy)-phenyl]-acetamide

C₂₈H₃₇N₅O₈ (571.6358)

ESI-TOF MS: 572 [M+H]

### Example 4

### 2-(3-Carbamimidoyl-phenylamino)-N-(4-phenoxy-phenyl)-2-[2-(3,4,5-trihydroxy-6-hydroxymethyl-tetrahydro-pyran-2-yloxy)-phenyl]-acetamide

C₃₃H₃₄N₄O₈ (614.6609)

ESI-TOF MS: 615 [M+H]

### Example 5

### 2-(3-Carbamimidoyl-phenylamino)-N-(3,3-diphenyl-propyl)-2-[2-(3,4,5-trihydroxy-6-hydroxymethyl-tetrahydro-pyran-2-yloxy)-phenyl]-acetamide

C₃₆H₄₀N₄O₇ (640.7428)

ESI-TOF MS: 641 [M+H]

### Example 6

### 2-(3-Carbamimidoyl-phenylamino)-N-(3-phenoxy-phenyl)-2-[2-(3,4,5-trihydroxy-6-hydroxymethyl-tetrahydro-pyran-2-yloxy)-phenyl]-acetamide

C₃₃H₃₄N₄O₈ (614.6609)

ESI-TOF MS: 615 [M+H]

### Example 7

### 2-(3-Carbamimidoyl-phenylamino)-N-(4-methoxy-biphenyl-3-yl)-2-[2-(3,4,5-trihydroxy-6-hydroxymethyl-tetrahydro-pyran-2-yloxy)-phenyl]-acetamide

C₃₄H₃₆N₄O₈ (628.6880)

ESI-TOF MS: 629 [M+H]

### Example 8

### 2-(3-Carbamimidoyl-phenylamino)-N-(4-morpholin-4-yl-phenyl)-2-[2-(3,4,5-trihydroxy-6-hydroxymethyl-tetrahydro-pyran-2-yloxy)-phenyl]-acetamide

C₃₁H₃₇N₅O₈ (607.6692)

ESI-TOF MS: 608 [M+H]

### Example 9

### 2-(3-Carbamimidoyl-phenylamino)-N-(4-benzoyl-phenyl)-2-[2-(3,4,5-trihydroxy-6-hydroxymethyl-tetrahydro-pyran-2-yloxy)-phenyl]-acetamide

C₃₄H₃₄N₄O₈ (626.6721)

ESI-TOF MS: 627 [M+H]

### Example 10

### 2-(3-Carbamimidoyl-phenylamino)-N-(3-benzoyl-phenyl)-2-[2-(3,4,5-trihydroxy-6-hydroxymethyl-tetrahydro-pyran-2-yloxy)-phenyl]-acetamide

C₃₄H₃₄N₄O₈ (626.6721)

ESI-TOF MS: 627 [M+H]

### Example 11

### 2-(3-Carbamimidoyl-phenylamino)-N-(4-tert-butyl-benzyl)-2-[2-(3,4,5-trihydroxy-6-hydroxymethyl-tetrahydro-pyran-2-yloxy)-phenyl]-acetamide

C₃₂H₄₀N₄O₇ (592.6982)

ESI-TOF MS: 593 [M+H]

### Example 12

### 2-(2-Hydroxy-5-carbamimidoyl-phenylamino)-N-(4-morpholin-4-yl-phenyl)-2-[2-(3,4,5-trihydroxy-6-hydroxymethyl-tetrahydro-pyran-2-yloxy)-phenyl]-acetamide

C₃₁H₃₇N₅O₉ (623.6686)

ESI-TOF MS: 624 [M+H]

### Example 13

### 2-(3-Carbamimidoyl-phenylamino)-N-(3-methoxy-benzyl)-2-[2-(3,4,5-trihydroxy-6-hydroxymethyl-tetrahydro-pyran-2-yloxy)-phenyl]-acetamide

C₂₉H₃₄N₄O₈ (566.6163)

ESI-TOF MS: 567 [M+H]

### Example 14

### N-(4-Acetyl-phenyl)-2-(3-carbamimidoyl-phenylamino)-2-[2-(3,4,5-trihydroxy-6-hydroxymethyl-tetrahydro-pyran-2-yloxy)-phenyl]-acetamide

C₂₉H₃₂N₄O₈ (564.6004)

ESI-TOF MS: 565 [M+H]

### Example 15

### 2-(3-Carbamimidoyl-phenylamino)-N-(3-trifluoromethyl-benzyl)-2-[2-(3,4,5-trihydroxy-6-hydroxymethyl-tetrahydro-pyran-2-yloxy)-phenyl]-acetamide

C₂₉H₃₁F₃N₄O₇ (604.5882)

ESI-TOF MS: 605 [M+H]

### Example 16

### 2-(3-Carbamimidoyl-phenylamino)-N-(2-cyclohex-1-enyl-ethyl)-2-[2-(3,4,5-trihydroxy-6-hydroxymethyl-tetrahydro-pyran-2-yloxy)-phenyl]-acetamide

C₂₉H₃₈N₄O₇ (554.6488)

ESI-TOF MS: 555 [M+H]

### Example 17

### 2-(3-Carbamimidoyl-phenylamino)-N-[2-(3,4-dimethoxy-phenyl)-ethyl]-2-[2-(3,4,5-trihydroxy-6-hydroxymethyl-tetrahydro-pyran-2-yloxy)-phenyl]-acetamide

C₃₁H₃₈N₄O₉ (610.6699)

ESI-TOF MS: 611 [M+H]

### Example 18

### 2-(3-Carbamimidoyl-phenylamino)-N-(3-morpholin-4-yl-propyl)-2-[2-(3,4,5-trihydroxy-6-hydroxymethyl-tetrahydro-pyran-2-yloxy)-phenyl]-acetamide

C₂₈H₃₉N₅O₈ (573.6517)

ESI-TOF MS: 574 [M+H]

### Example 19

### 2-(3-Carbamimidoyl-phenylamino)-N-(4-trifluoromethyl-benzyl)-2-[2-(3,4,5-trihydroxy-6-hydroxymethyl-tetrahydro-pyran-2-yloxy)-phenyl]-acetamide

C₂₉H₃₁F₃N₄O₇ (604.5882)

ESI-TOF MS: 605 [M+H]

### Example 20

### N-[1-(4-Bromo-phenyl)-ethyl]-2-(3-carbamimidoyl-phenylamino)-2-[2-(3,4,5-trihydroxy-6-hydroxymethyl-tetrahydro-pyran-2-yloxy)-phenyl]-acetamide

C₂₉H₃₃BrN₄O₇ (629.5130)

ESI-TOF MS: 630 [M+H]

### Example 21

### N-Benzo[1,3]dioxol-5-ylmethyl-2-(3-carbamimidoyl-phenylamino)-2-[2-(3,4,5-trihydroxy-6-hydroxymethyl-tetrahydro-pyran-2-yloxy)-phenyl]-acetamide

C₂₉H₃₂N₄O₉ (580.5998)

ESI-TOF MS: 581 [M+H]

### Example 22

### 2-(3-Carbamimidoyl-phenylamino)-N-(3-phenyl-propyl)-2-[2-(3,4,5-trihydroxy-6-hydroxymethyl-tetrahydro-pyran-2-yloxy)-phenyl]-acetamide

C₃₀H₃₆N₄O₇ (564.6440)

ESI-TOF MS: 565 [M+H]

### Example 23

### 2-(3-Carbamimidoyl-phenylamino)-N-(3,5-dimethyl-benzyl)-2-[2-(3,4,5-trihydroxy-6-hydroxymethyl-tetrahydro-pyran-2-yloxy)-phenyl]-acetamide

C₃₀H₃₆N₄O₇ (564.6440)

ESI-TOF MS: 565 [M+H]

### Example 24

### 2-(3-Carbamimidoyl-phenylamino)-N-(3-cyano-phenyl)-2-[2-(3,4,5-trihydroxy-6-hydroxymethyl-tetrahydro-pyran-2-yloxy)-phenyl]-acetamide

C₂₈H₂₉N₅O₇ (547.5726)

ESI-TOF MS: 548 [M+H]

### Example 25

### 2-(3-Carbamimidoyl-phenylamino)-N-(3,4-dichloro-benzyl)-2-[2-(3,4,5-trihydroxy-6-hydroxymethyl-tetrahydro-pyran-2-yloxy)-phenyl]-acetamide

C₂₈H₃₀Cl₂N₄O₇ (605.4799)

ESI-TOF MS: 606 [M+H]

### Example 26

### N-(3-Acetyl-phenyl)-2-(3-carbamimidoyl-phenylamino)-2-[2-(3,4,5-trihydroxy-6-hydroxymethyl-tetrahydro-pyran-2-yloxy)-phenyl]-acetamide

C₂₉H₃₂N₄O₈ (564.6004)

ESI-TOF MS: 565 [M+H]

### Example 27

### 2-(3-Carbamimidoyl-phenylamino)-2-[2-(3,4,5-trihydroxy-6-hydroxymethyl-tetrahydro-pyran-2-yloxy)-phenyl]-N-(1,2,2-trimethyl-propyl)-acetamide

C₂₇H₃₈N₄O₇ (530.6265)

ESI-TOF MS: 531 [M+H]

### Example 28

### N-Allyl-2-(3-carbamimidoyl-phenylamino)-2-[2-(3,4,5-trihydroxy-6-hydroxymethyl-tetrahydro-pyran-2-yloxy)-phenyl]-acetamide

C₂₄H₃₀N₄O₇ (486.5293)

ESI-TOF MS: 487 [M+H]

### Example 29

### N-(3-Butoxy-propyl)-2-(3-carbamimidoyl-phenylamino)-2-[2-(3,4,5-trihydroxy-6-hydroxymethyl-tetrahydro-pyran-2-yloxy)-phenyl]-acetamide

C₂₈H₄₀N₄O₈ (560.6530)

ESI-TOF MS: 561 [M+H]

### Example 30

### 2-(3-Carbamimidoyl-phenylamino)-N-(3,7-dimethyl-octa-2,6-dienyl)-2-[2-(3,4,5-trihydroxy-6-hydroxymethyl-tetrahydro-pyran-2-yloxy)-phenyl]-acetamide

C₃₁H₄₂N₄O₇ (582.7030)

ESI-TOF MS: 583 [M+H]

### Example 31

### 2-(3-Carbamimidoyl-phenylamino)-N-furan-2-ylmethyl-2-[2-(3,4,5-trihydroxy-6-hydroxymethyl-tetrahydro-pyran-2-yloxy)-phenyl]-acetamide

C₂₆H₃₀N₄O₈ (526.5510)

ESI-TOF MS: 527 [M+H]

### Example 32

### 2-(3-Carbamimidoyl-phenylamino)-N-(3-isopropoxy-propyl)-2-[2-(3,4,5-trihydroxy-6-hydroxymethyl-tetrahydro-pyran-2-yloxy)-phenyl]-acetamide

C₂₇H₃₈N₄O₈ (546.6259)

ESI-TOF MS: 547 [M+H]

### Example 33

### 3-{2-(3-Carbamimidoyl-phenylamino)-2-[2-(3,4,5-trihydroxy-6-hydroxymethyl-tetrahydro-pyran-2-yloxy)-phenyl]-acetylamino}-propionic acid ethyl ester

C₂₆H₃₄N₄O₉ (546.5823)

ESI-TOF MS: 547 [M+H]

### Example 34

### N-tert-Butyl-2-(3-carbamimidoyl-phenylamino)-2-[2-(3,4,5-trihydroxy-6-hydroxymethyl-tetrahydro-pyran-2-yloxy)-phenyl]-acetamide

C₂₅H₃₄N₄O₇ (502.5723)

ESI-TOF MS: 503 [M+H]

### Example 35

### 2-(3-Carbamimidoyl-phenylamino)-N-pyridin-4-ylmethyl-2-[2-(3,4,5-trihydroxy-6-hydroxymethyl-tetrahydro-pyran-2-yloxy)-phenyl]-acetamide

C₂₇H₃₁N₅O₇ (537.5774)

ESI-TOF MS: 538 [M+H]

### Example 36

### 2-(3-Carbamimidoyl-phenylamino)-N-methyl-2-[2-(3,4,5-trihydroxy-6-hydroxymethyl-tetrahydro-pyran-2-yloxy)-phenyl]-acetamide

C₂₂H₂₈N₄O₇ (460.4911)

ESI-TOF MS: 461 [M+H]

### Example 37

### 2-(3-Carbamimidoyl-phenylamino)-N-(1,3-dimethyl-butyl)-2-[2-(3,4,5-trihydroxy-6-hydroxymethyl-tetrahydro-pyran-2-yloxy)-phenyl]-acetamide

C₂₇H₃₈N₄O₇ (530.6265)

ESI-TOF MS: 531 [M+H]

### Example 38

### 2-(3-Carbamimidoyl-phenylamino)-N-(4-morpholin-4-yl-phenyl)-2-phenyl-acetamide

C₂₅H₂₇N₅O₂ (429.5262)

ESI-TOF MS: 430 [M+H]

### Example 39

### 2-(3-Carbamimidoyl-phenylamino)-2-phenyl-N-(2′-trifluoromethyl-biphenyl-4-yl)-acetamide

C₂₈H₂₃F₃N₄O (488.5169)

ESI-TOF MS: 489 [M+H]

### Example 40

### N-(1-Benzyl-piperidin-4-yl)-2-(3-carbamimidoyl-phenylamino)-2-phenyl-acetamide

C₂₇H₃₁N₅O (441.5810)

ESI-TOF MS: 442 [M+H]

### Example 41

### 2-(3-Carbamimidoyl-phenylamino)-N-[4-(morpholin-4-carbonyl)-phenyl]-2-phenyl-acetamide

C₂₆H₂₇N₅O₃ (457.5368)

ESI-TOF MS: 458 [M+H]

### Example 42

### {2-[(3-Carbamimidoyl-phenylamino)-(4-morpholin-4-yl-phenylcarbamoyl)-methyl]-phenoxy}-acetic acid

C₂₇H₂₉N₅O₅ (503.5627)

ESI-TOF MS: 503 [M+H]

### Example 43

### {3-[(3-Carbamimidoyl-phenylamino)-(4-morpholin-4-yl-phenylcarbamoyl)-methyl]-phenoxy}-acetic acid

C₂₇H₂₉N₅O₅ (503.5627)

ESI-TOF MS: 503 [M+H]

### Example 44

### {2-[(3-Carbamimidoyl-phenylamino)-(4-morpholin-4-yl-phenylcarbamoyl)-methyl]-phenoxy}-acetic acid methyl ester

C₂₈H₃₁N₅O₅ (517.5898)

ESI-TOF MS: 518 [M+H]

### Example 45

### {3-[(3-Carbamimidoyl-phenylamino)-(4-morpholin-4-yl-phenylcarbamoyl)-methyl]-phenoxy}-acetic acid methyl ester

C₂₈H₃₁N₅O₅ (517.5898)

ESI-TOF MS: 518 [M+H]

### Example 46

### {2-[(4-Benzoyl-phenylcarbamoyl)-(3-carbamimidoyl-phenylamino)-methyl]-phenoxy}-acetic acid methyl ester

C₃₁H₂₈N₄O₅ (536.5926)

ESI-TOF MS: 537 [M+H]

### Example 47

### {3-[(4-Benzoyl-phenylcarbamoyl)-(3-carbamimidoyl-phenylamino)-methyl]-phenoxy}-acetic acid methyl ester

C₃₁H₂₈N₄O₅ (536.5926)

ESI-TOF MS: 537 [M+H]

### Example 48

### {2-[(4-Benzoyl-phenylcarbamoyl)-(3-carbamimidoyl-phenylamino)-methyl]-phenoxy}-acetic acid

C₃₀H₂₆N₄O₅ (522.5655)

ESI-TOF MS: 523 [M+H]

### Example 49

### {3-[(4-Benzoyl-phenylcarbamoyl)-(3-carbamimidoyl-phenylamino)-methyl]-phenoxy}-acetic acid

C₃₀H₂₆N₄O₅ (522.5655)

ESI-TOF MS: 523 [M+H]

### Example 50

### (2-((3-Carbamimidoyl-phenylamino)-[4-(morpholine-4-carbonyl)-phenylcarbamoyl]-methyl}-phenoxy)-acetic acid methyl ester

C₂₉H₃₁N₅O₆ (545.6003)

ESI-TOF MS: 546 [M+H]

### Example 51

### (3-{(3-Carbamimidoyl-phenylamino)-[4-(morpholine-4-carbonyl)-phenylcarbamoyl]-methyl}-phenoxy)-acetic acid methyl ester

C₂₉H₃₁N₅O₆ (545.6003)

ESI-TOF MS: 546 [M+H]

### Example 52

### (2-{(3-Carbamimidoyl-phenylamino)-[4-(morpholine-4-carbonyl)-phenylcarbamoyl]-methyl}-phenoxy)-acetic acid

C₂₈H₂₉N₅O₆ (531.5732)

ESI-TOF MS: 532 [M+H]

### Example 53

### (3-{(3-Carbamimidoyl-phenylamino)-[4-(morpholine-4-carbonyl)-phenylcarbamoyl]-methyl}-phenoxy)-acetic acid

C₂₈H₂₉N₅O₆ (531.5732)

ESI-TOF MS: 532 [M+H]

In order to demonstrate the inhibitory action towards factor Xa activity, chromogenic peptide substrates were used. The inhibition of the amidolytic activity of factor Xa by the compounds described above was demonstrated as follows. The measurements were carried out in micro-titre plates at room temperature. The compounds were dissolved in dimethyl sulphoxide and 5 μl of the solution were added to a 1 nM solution of human recombinant factor Xa (Enzyme Research Laboratories, South Bend, Ind., USA) in a buffer (pH: 8.0 and using 50 mM Tris-HCl, 100 mM NaCl, 0.1% PEG 6000 and 0.05% Tween 80). Finally, 200 μM N-methoxy-carbonyl-D-norleucyl-glycyl-L-arginine-4-nitranilide acetate (Roche Diagnostics, Mannheim, Germany) in buffer were added and the hydrolysis of the substrate was monitored with a Spectra Flour Plus spectrophotometer (Tecan, Crailsheim, Germany) over a period of 20 minutes. The IC₅₀ values were calculated by means of the “GraFit 4” program of the company Erithacus Software Ltd. (Staines, Middlesex, UK). On the assumption that the kinetics comprise a competitive inhibition, it was possible to determine the Kᵢ value by the Cheng-Prusoff equation: Kᵢ=IC₅₀/(1+[S]/Kₘ]) (Cheng and Prusoff, Biochemical Pharmacology 1973, 22: 3099-3108). The same procedure, but with tosyl-glycyl-prolyl-lysine-4-nitranilide acetate being used as the substrate in Hepes buffer (pH 7.8), was used to determine the inhibition of the proteolytic activity of recombinant human tryptase (Promega, Madison, Wis., USA) by the said compounds.

The IC₅₀ values of the above-mentioned Examples are in the range from 1 nM to 1 μM.

1. A compound comprising formula (I): 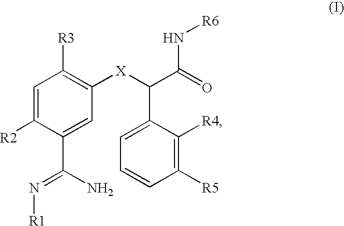
wherein
R1 is a hydrogen atom, a heteroalkyl, heteroaralkyl, heterocycloalkyl, hydroxy or alkyloxy group, R2 is a hydrogen atom or a hydroxy group, or R1 and R2 together are part of a 5- or 6-membered ring;
R3 is a hydrogen atom, a hydroxy, alkyloxy, amino, alkylamino or dialkylamino group or a halogen atom;
R4 and R5 are, each independently of the other, a hydrogen atom, a halogen atom, a hydroxy, amino, nitro or thiol group, an alkyl, heteroalkyl, aryl, heteroaryl, cycloalkyl, heterocycloalkyl, aralkyl or heteroaralkyl radical or a glycosyloxy group;
R6 is an alkyl, heteroalkyl, heteroaralkyl, heteroaryl, aralkyl, cycloalkyl, heterocycloalkyl or aryl group, R6 not being a group of formula —CHR8-CO—NR9R9′, wherein R8, R9 and
R9′ are, each independently of the others, a hydrogen atom, an alkyl, heteroalkyl, heteroaralkyl, heteroaryl, aralkyl, cycloalkyl, heterocycloalkyl or aryl group, or R9 and R9′ together are part of a heterocycloalkyl or heteroaryl ring system, and
X is a group of formula NR7, O, S, SO, SO₂, SO₂NH, PO₂NH, CH₂, CHMe or CO, R7 being a hydrogen atom, an alkyl or aralkyl group;
or a pharmacologically acceptable salt, solvate, hydrate or pharmacologically acceptable formulation thereof.

2. Compounds according to claim 1, wherein R1 is a hydrogen atom.

3. Compounds according to claim 1, wherein R2 is a hydrogen atom.

4. Compounds according to claim 1, wherein R3 is a hydrogen atom or a hydroxy group.

5. Compounds according to claim 1, wherein R4 is a hydrogen atom, an —OH, —OCH₂COOH, —COOH, —OCH₂COOCH₃, C₁-C₄alkyloxy or glycosyloxy group or a halogen atom.

6. Compounds according to claim 1, wherein R4 is a β-D-glucosyloxy group.

7. Compounds according to claim 1, wherein R5 is a hydrogen atom, an —OH, —OCH₂COOH, —COOH, —OCH₂COOCH₃, C₁-C₄alkyloxy or glycosyloxy group or a halogen atom.

8. Compounds according to claim 1, wherein R5 is a hydrogen atom.

9. Compounds according to claim 1 , wherein X is an NH group.

10. Pharmaceutical compositions comprising a compound according to claim 1 as active ingredient and, optionally, carrier substances and/or adjuvants.

11. A method for inhibiting factor Xa by administering a compound of claim 1.

12. A method for the treatment or prevention of thromboembolic conditions, arterial restenosis, septicaemia, cancer, acute inflammation or other conditions mediated by factor Xa activity comprising administering a compound of claim 1.

13. A method for preventing or reducing complications relating to thromboembolic conditions in vascular surgery comprising administering a compound of claim 1 prior to, during or following said surgery.
